(19) United States
(12) Patent Application Publication
(10) Pub. No.: US 2003/0040540 A1
(43) Pub. Date: Feb. 27, 2003

(54) Bromonitrothienyldioxanes

(76) Inventors: Hermann Uhr, Leverkusen (DE); Karl-Heinz Kuck, Langenfeld (DE); Martin Kugler, Leichlingen (DE); Peter Wachtler, Krefeld (DE)

Correspondence Address: BAYER CORPORATION, PATENT DEPARTMENT, 100 BAYER ROAD, PITTSBURGH, PA, 15205, US

(21) Appl. No.: 10/189,934

(22) Filed: Jul. 3, 2002

(30) Foreign Application Priority Data
Jul. 9, 2001 (DE) 10133276.9

(51) Int. Cl. C 07D 4 9/04; A61K 31/381

(52) U.S. Cl. 514/444; 549/60

(57) ABSTRACT

The invention relates to novel 5-bromo-5-nitro-2-thienyl-1,3-dioxanes of the formula (I)

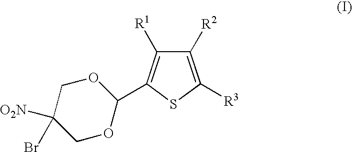

in which

R¹, R² and R³ are as defined in the description are highly suitable for use as biocides for protecting plants and industrial materials.

### BACKGROUND OF THE INVENTION

The present invention relates to novel 5-bromo-5-nitro-2-thienyl-1,3-dioxanes, to processes for their preparation and to the use of novel and known bromonitrothienyldioxanes as fungicides and bactericides for protecting industrial materials and plants.

Bromonitrodioxanes having antibacterial and antifungal action are already known.

The most simple representative 5-bromo-5-nitro-1,3-dioxane (bromidox) is used for preserving cosmetics (cf. U.S. Pat. No. 3,772,443).

5-Nitrofural acetals, inter alia the structurally similar 5-bromo-5-nitro-2-(5-nitro-2-furyl)-1,3-dioxane are described in DE-A-2237053 as antimicrobial substances for preserving cosmetics and industrial materials.

JP-A-59084881 describes antibacterial and antifungal bronopol acetals with benzaldehydes and cinnamaldehydes.

With respect to their microbicidal properties, the 5-bromo-5-nitrodioxanes known from the prior art still do not meet all the requirements they ought to meet. Surprisingly, we have found novel 5-bromo-5-nitro-2-thienyl-1,3-dioxanes which are highly suitable for use as microbicides for protecting plants and industrial materials and which have an action which is clearly superior to the prior art.

### SUMMARY OF THE INVENTION

The invention relates to a 5-bromo-5-nitro-2-thienyl-1,3-dioxane of the formula (I)

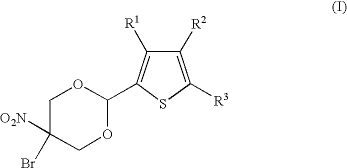

wherein R¹, R² and R³ independently of one another represent hydrogen, halogen, nitro, cyano, or represent in each case an optionally substituted alkyl, alkenyl, alkynyl, acyl, acyloxy, alkoxy, alkoxycarbonyl, alkylthio, amino, mono- or dialkylamino group.

The invention also relates to a process for preparing a 5-bromo-5-nitro-2-thienyl-1,3-dioxane of the formula (I), in a process that involves A) reacting, in a transacetalization reaction, 2-bromo-2-nitro-propane-1,3-diol (bronopol) with an acetal of the general formula (III)

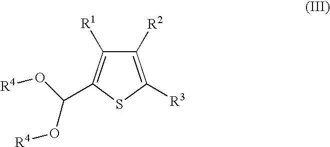

wherein R¹, R² and R³ are independently of one another represent hydrogen, halogen, nitro, cyano, or represent in each case an optionally substituted alkyl, alkenyl, alkynyl, acyl, acyloxy, alkoxy, alkoxycarbonyl, alkylthio, amino, mono- or dialkylamino group, and R⁴ represents alkyl, optionally in the presence of a catalyst and optionally, in the presence of a diluent.

The invention also relates to a compound of the formula (III):

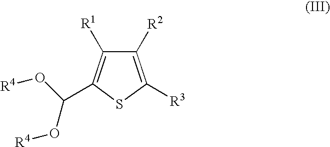

wherein R¹, R² and R³ are independently of one another represent hydrogen, halogen, nitro, cyano, or represent in each case an optionally substituted alkyl, alkenyl, alkynyl, acyl, acyloxy, alkoxy, alkoxycarbonyl, alkylthio, amino, mono- or dialkylamino group, and R⁴ represents alkyl.

The invention also relates to a process for preparing a compound of formula (III), in which aldehydes of the general formula (II)

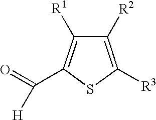

wherein R¹, R² and R³ are independently of one another represent hydrogen, halogen, nitro, cyano, or represent in each case an optionally substituted alkyl, alkenyl, alkynyl, acyl, acyloxy, alkoxy, alkoxycarbonyl, alkylthio, amino, mono- or dialkylamino group, react with an alcohol of the formula (IV),

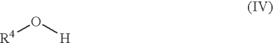

in which R⁴ represents alkyl, optionally in the presence of a catalyst, optionally in the presence of a dehydrating agent if appropriate, in a diluent.

The invention also relates to microbicidal compositions containing a compound of formula (I) and a method for protecting an industrial material or a plant, a seed, or soil with the compound of formula (I).

### DETAILED DESCRIPTION OF THE INVENTION

The present invention provides 5-bromo-5-nitro-2-thienyl-1,3-dioxanes of the general formula (I),

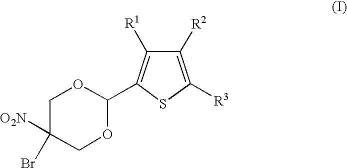

in which R¹, R² and R³ independently of one another represent hydrogen, halogen, nitro, cyano, or represent in each case optionally substituted alkyl, alkenyl, alkynyl, acyl, acyloxy, alkoxy, alkoxycarbonyl, alkylthio, amino, mono- or dialkylamino.

The formula (I) provides a general definition of the compounds according to the invention. Preference is given to compounds of the formula (I), in which R¹, R² and R³ independently of one another represent hydrogen, halogen, cyano, nitro, or represent in each case straight-chain or branched C₁-C₈-alkyl, C₂-C₈-alkenyl or C₂-C₈-alkynyl which are in each case optionally mono- or polysubstituted by identical or different substitutents from the group consisting of halogen; nitro; cyano; C₁-C₆-alkoxy or C₁-C₆-alkylthio which are in each case optionally mono- to nonasubstituted by identical or different halogens, or represent C₁-C₆-acyl, represent C₁-C₆-acyloxy, represent C₁-C₆-alkoxycarbonyl which is optionally mono- or disubstituted by identical or different halogens, represent C₁-C₅-alkoxy or C₁-C₅-alkylthio which are in each case optionally mono- to hexasubstituted by identical or different halogens, or represent amino, monoalkylamino having straight-chain or branched C₁-C₆-alkyl radicals or represent dialkylamino having identical or different straight-chain or branched C₁-C₆-alkyl radicals.

Particular preference is given to compounds of the formula (I) in which R¹, R² and R³ independently of one another represent hydrogen, fluorine, chlorine, bromine, cyano, nitro, or represent in each case straight-chain or branched C₁-C₆-alkyl, C₂-C₆-alkenyl or C₂-C₆-alkynyl which are in each case optionally mono- to tetrasubstituted by identical or different substituents from the group consisting of fluorine; chlorine; bromine; nitro; cyano; C₁-C₄-alkoxy or C₁-C₄-alkylthio which are in each case optionally mono- to heptasubstituted by identical or different substituents from the group consisting of fluorine, chlorine and bromine, or represent C₁-C₄-acyl, represent C₁-C₄-acyloxy, represent C₂-C₄-alkoxycarbonyl which is optionally mono- or disubstituted by identical or different substituents from the group consisting of fluorine, chlorine and bromine, represent C₁-C₄-alkoxy, or C₁-C₄-alkylthio, each of which is optionally mono- to tetrasubstituted by identical or different substituents from the group consisting of fluorine, chlorine and bromine, or represent amino, monoalkylamino having straight-chain or branched C₁-C₄-alkyl radicals or represent dialkylamino having identical or different straight-chain or branched C₁-C₄-alkyl radicals.

Very particularly preferred are compounds of the formula (I) in which

R¹, R² and R³ independently of one another represent hydrogen, fluorine, chlorine, bromine, cyano, nitro, or represent methyl, ethyl, n-propyl, isopropyl, n-butyl, isobutyl, sec-butyl, tert-butyl, n-pentyl, allyl, vinyl, propargyl, each of which is optionally mono- to tetrasubstituted by identical or different substituents from the group consisting of fluorine; chlorine; bromine; nitro; cyano; methoxy; ethoxy; n-propoxy; isopropoxy; n-butoxy; isobutyoxy; sec-butoxy; tert-butoxy; trifluoromethoxy; methylthio; ethylthio; n-propylthio; isopropylthio; trifluoromethylthio, or represent formyl, acetyl, propionyl, formyloxy, acetoxy, propionyloxy, methyl carboxylate, ethyl carboxylate, methoxy, ethoxy, n-propoxy, isopropoxy, trifluoromethoxy, methylthio, ethylthio, n-propylthio, isopropylthio, trifluoromethylthio, amino, methylamino, ethylamino, n-propylamino, isopropylamino, dimethylamino, diethylamino, methylethylamino, di-n-propylamino or diisopropylamino.

The radicals given in the respective definitions or preferred or particularly preferred definitions can, independently of the combination given in each case, also be replaced by any radical definitions of other combinations. Moreover, radical definitions from any preferred range may not apply.

The present invention furthermore relates to processes for preparing the compounds of the formula (I) according to the invention, by reacting 2-bromo-2-nitropropane-1,3-diol (bronopol) with an aldehyde of the general formula (II)

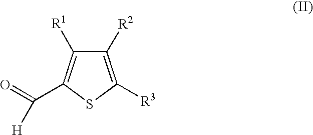

in which R¹, R² and R³ are as defined above, if appropriate in the presence of a catalyst, if appropriate in the presence of a dehydrating agent and, if appropriate, in a diluent.

Suitable catalysts are all catalysts which promote the acetalization. In general, use is made of acids or Lewis acids. Preference is given to using inorganic acids, such as HCl, H₂SO₄ or H₃PO₄, organic acids, such as formic acid, acetic acid or p-toluenesulfonic acid, or Lewis acids, such as AlCl₃, ZnCl₂, TiCl₄ or ClSi(CH₃)₃.

Suitable for use as dehydrating agents are all compounds capable of binding water or eliminating it by chemical reaction. Preferred dehydrating agents are Na₂SO₄, MgSO₄, CaCl₂, molecular sieves, dried aluminium oxide, TiCl₄, ClSi(CH₃)₃ and P₂O₅. However, it is also possible to remove the water of reaction formed azeotropically from the reaction mixture using a solvent.

Suitable solvents which are used, if appropriate, are all organic solvents. These preferably include hydrocarbons, such as toluene, xylene or hexane, chlorinated hydrocarbons, such as chlorobenzene, methylene chloride or chloroform, ethers, such as tetrahydrofuran, diethyl ether, methyl tert-butyl ether and dioxane, nitriles, such as acetonitrile, and also DMSO, DMF and NMP.

In this process, the reaction temperatures can be varied within a relatively wide temperature range. In general, the process is carried out between 10° C. and +180° C., preferably between 20° C. and +145° C.

The compounds of the general formula (II) are known and generally commercially available.

Alternatively, the compounds of the formula (I) according to the invention can be prepared by reacting 2-bromo-2-nitropropane-1,3-diol

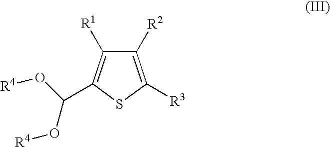

(bronopol) in a transacetalization reaction with an acetal of the general formula (III)

in which

R¹, R² and R³ are as defined above and

R⁴ represents alkyl, in particular methyl, ethyl, n-propyl or isopropyl, if appropriate in the presence of a catalyst and, if appropriate, in the presence of a diluent.

Suitable for use as catalysts are all catalysts which promote the transacetalization. In general, acids or Lewis acids are employed. Preference is given to using inorganic acids, such as HCl, H₂SO₄ or H₃PO₄, organic acids, such as formic acid, acetic acid or p-toluenesulfonic acid, or Lewis acids such as AlCl₃, ZnCl₂ or TiCl₄.

Suitable solvents which are employed, if appropriate, are all organic solvents. These preferably include hydrocarbons, such as toluene, xylene or hexane, chlorinated hydrocarbons, such as chlorobenzene, methylene chloride or chloroform, ethers, such as tetrahydrofuran, diethyl ether, methyl tert-butyl ether and dioxane, nitriles, such as acetonitrile, and also DMSO, DMF and NMP.

The compounds of the general formula (III) are novel and also form part of the subject-matter of the present invention. The novel compounds (III) can be prepared by reacting aldehydes of the general formula (II) with an alcohol of the formula (IV)

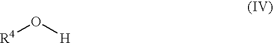

in which

R⁴ is as defined above, if appropriate in the presence of a catalyst, if appropriate in the presence of a dehydrating agent and if appropriate in a diluent. If appropriate, the reaction can also be carried out under superatmospheric pressure in an autoclave.

Suitable for use as catalysts are all catalysts which promote the acetalization. In general, acids or Lewis acids are employed. Preference is given to using inorganic acids, such as HCl, H₂SO₄ or H₃PO₄, organic acids, such as formic acid, acetic acid or p-toluenesulfonic acid, or Lewis acids, such as AlCl₃, ZnCl₂, TiCl₄ or ClSi(CH₃)₃.

Suitable for use as dehydrating agents are all compounds capable of binding water or removing it by chemical reaction. Preferred dehydrating agents are Na₂SO₄, MgSO₄, CaCl₂, molecular sieves, dried aluminium oxide, TiCl₄, ClSi(CH₃)₃ or P₂O₅. However, it is also possible to remove the water of reaction formed azeotropically from the reaction mixture using a solvent.

Suitable solvents which are employed, if appropriate, are all organic solvents. These preferably include hydrocarbons, such as toluene, xylene or hexane, chlorinated hydrocarbons, such as chlorobenzene, methylene chloride or chloroform, ethers, such as tetrahydrofuran, diethyl ether, methyl tert-butyl ether and dioxane, nitriles, such as acetonitrile, and also DMSO, DMF and NMP.

In this process, the reaction temperatures can be varied within a relatively wide temperature range. In general, the process is carried out between 10° C. and +180° C., preferably between 20° C. and +145° C.

The compounds of the formula (I) according to the invention have potent microbicidal activity and can be employed for controlling undesirable microorganisms, such as fungi and bacteria, in crop protection and in the protection of materials.

Moreover, the compounds of the formula (I) have good long-term action and stability in industrial materials.

Accordingly, the present invention furthermore relates to the use of compounds of the formula (I) as microbicides for protecting industrial materials and plants against attack and destruction by microorganisms.

Fungicides can be employed in crop protection for controlling Plasmodiophoromycetes, Oomycetes, Chytridiomycetes, Zygomycetes, Ascomycetes, Basidiomycetes and Deuteromycetes.

Bactericides can be employed in crop protection for controlling Pseudomonadaceae, Rhizobiaceae, Enterobacteriaceae, Corynebacteriaceae and Streptomycetaceae.

Some pathogens causing fungal and bacterial diseases which come under the generic names listed above are mentioned as examples, but not by way of limitation:

Xanthomonas species, such as Xanthomonas campestris pv. oryzae; Pseudomonas species, such as Pseudomonas syringae pv. lachrymans; Erwinia species, such as Erwinia amylovora; Pythium species, such as Pythium ultimum; Phytophthora species, such as Phytophthora infestans; Pseudoperonospora species, such as Pseudoperonospora humuli or Pseudoperonospora cubensis; Plasmopara species, such as Plasmopara viticola; Bremia species, such as Bremia lactucae; Peronospora species, such as Peronospora pisi or P. brassicae;

Erysiphe species, such as Erysiphe graminis; Sphaerotheca species, such as Sphaerotheca fuliginea; Podosphaera species, such as Podosphaera leucotricha; Venturia species, such as Venturia inaequalis; Pyrenophora species, such as Pyrenophora teres or P. graminea (conidia form: Drechslera, syn: Helminthosporium); Cochliobolus species, such as Cochliobolus sativus (conidia form: Drechslera, syn: Helminthosporium); Uromyces species, such as Uromyces appendiculatus; Puccinia species, such as Puccinia recondita; Sclerotinia species, such as Sclerotinia sclerotiorum; Tilletia species, such as Tilletia caries; Ustilago species, such as Ustilago nuda or Ustilago avenae; Pellicularia species, such as Pellicularia sasakii; Pyricularia species, such as Pyricularia oryzae; Fusarium species, such as Fusarium culmorum; Botrytis species, such as Botrytis cinerea; Septoria species, such as Septoria nodorum; Leptosphaeria species, such as Leptosphaeria nodorum; Cercospora species, such as Cercospora canescens; Alternaria species, such as Alternaria brassicae; and Pseudocercosporella species, such as Pseudocercosporella herpotrichoides.

The fact that the active compounds are well tolerated by plants at the concentrations required for controlling plant diseases permits the treatment of aerial parts of plants, of propagation stock and seeds, and of the soil.

Furthermore, the compounds according to the invention may also be employed to increase the yield of crops. Moreover, they have reduced toxicity and are tolerated well by plants.

In the protection of materials, the compounds according to the invention can be employed for protecting industrial materials against infection with, and destruction by, undesired microorganisms.

Industrial materials in the present context are understood as meaning non-living materials which have been prepared for use in industry. For example, industrial materials which are intended to be protected by active compounds according to the invention from microbial change or destruction can be adhesives, sizes, paper and board, textiles, leather, wood, paints and plastic articles, cooling lubricants and other materials which can be infected with, or destroyed by, microorganisms. Parts of production plants, for example cooling-water circuits, which may be impaired by the proliferation of microorganisms may also be mentioned within the scope of the materials to be protected. Industrial materials which may be mentioned within the scope of the present invention are preferably adhesives, sizes, paper and board, leather, wood, paints, cooling lubricants and heat-transfer liquids.

Microorganisms capable of degrading or changing the industrial materials which may be mentioned are, for example, bacteria, fungi, yeasts, algae and slime organisms. The active compounds according to the invention preferably act against fungi, in particular moulds, wood-discolorizing and wood-destroying fungi (Basidiomycetes), and against slime organisms and algae.

Microorganisms of the following genera may be mentioned as examples: Alternaria, such as Alternaria tenuis, Aspergillus, such as Aspergillus niger, Chaetomium, such as Chaetomium globosum, Coniophora, such as Coniophora puetana, Lentinus, such as Lentinus tigrinus, Penicillium, such as Penicillium glaucum, Polyporus, such as Polyporus versicolor, Aureobasidium, such as Aureobasidium pullulans, Sclerophoma, such as Sclerophoma pityophila, Trichoderma, such as Trichoderma viride, Escherichia, such as Escherichia coli, Pseudomonas, such as Pseudomonas aeruginosa, and Staphylococcus, such as Staphylococcus aureus.

The present invention furthermore relates to microbicidal compositions comprising at least one compound of the formula (I), solvents or binders and, if appropriate, processing auxiliaries and, if appropriate, further active compounds.

Depending on their particular physical and/or chemical properties, the active compounds of the formula (I) can be converted to the customary formulations, such as solutions, emulsions, suspensions, powders, foams, pastes, granules, aerosols and microencapsulations in polymeric substances and in coating compositions for seeds, and ULV cool and warm fogging formulations.

These formulations are produced in a known manner, for example by mixing the active compounds with extenders, that is, liquid solvents, liquefied gases under pressure, and/or solid carriers, optionally with the use of surfactants, that is emulsifiers and/or dispersants, and/or foam formers. If the extender used is water, it is also possible to use for example organic solvents as auxiliary solvents. The following are mainly suitable as liquid solvents: aromatics such as xylene, toluene or alkylnaphthalenes, chlorinated aromatics or chlorinated aliphatic hydrocarbons such as chlorobenzenes, chloroethylenes or methylene chloride, aliphatic hydrocarbons such as cyclohexane or paraffins, for example petroleum fractions, alcohols such as butanol or glycol and their ethers and esters, ketones such as acetone, methyl ethyl ketone, methyl isobutyl ketone or cyclohexanone, strongly polar solvents such as dimethylformamide or dimethyl sulfoxide, or else water. Liquefied gaseous extenders or carriers are to be understood as meaning liquids which are gaseous at standard temperature and under atmospheric pressure, for example aerosol propellants such as halogenated hydrocarbons, or else butane, propane, nitrogen and carbon dioxide. Suitable solid carriers are: for example ground natural minerals such as kaolins, clays, talc, chalk, quartz, attapulgite, montmorillonite or diatomaceous earth, and ground synthetic minerals such as highly disperse silica, alumina and silicates. Suitable solid carriers for granules are: for example crushed and fractionated natural rocks such as calcite, marble, pumice, sepiolite and dolomite, or else synthetic granules of inorganic and organic meals, and granules of organic material such as sawdust, coconut shells, maize cobs and tobacco stalks. Suitable emulsifiers and/or foam formers are: for example nonionic and anionic emulsifiers, such as polyoxyethylene fatty acid esters, polyoxyethylene fatty alcohol ethers, for example alkylaryl polyglycol ethers, alkylsulfonates, alkyl sulfates, arylsulfonates, or else protein hydrolysates. Suitable dispersants are: for example lignin-sulfite waste liquors and methylcellulose.

Tackifiers such as carboxymethylcellulose and natural and synthetic polymers in the form of powders, granules or latices, such as gum arabic, polyvinyl alcohol and polyvinyl acetate, or else natural phospholipids such as cephalins and lecithins and synthetic phospholipids can be used in the formulations. Other additives can be mineral and vegetable oils.

It is possible to use colorants such as inorganic pigments, for example iron oxide, titanium oxide and Prussian Blue, and organic dyestuffs such as alizarin dyestuffs, azo dyestuffs and metal phthalocyanine dyestuffs, and trace nutrients such as salts of iron, manganese, boron, copper, cobalt, molybdenum and zinc.

The formulations generally comprise from about 0.1 to about 95 percent by weight of active compound, preferably from about 0.5 to about 90%.

The active compounds according to the invention can be used as such or in their formulations also mixed with known fungicides, bactericides, acaricides, nematicides or insecticides in order thus, for example, to widen the spectrum of action or to prevent development of resistance. In many cases, synergistic effects are achieved, i.e. the activity of the mixture exceeds the activity of the individual components.

Accordingly, the invention also relates to compositions comprising at least one compound of the formula (I) and at least one further antimicrobially active compound from the group of the fungicides, bactericides, acaricides, nematicides, algicides and/or insecticides.

Examples of co-components in plant protection applications in mixtures are the following compounds:

Fungicides:

aldimorph, ampropylfos, ampropylfos potassium, andoprim, anilazine, azaconazole, azoxystrobin, benalaxyl, benodanil, benomyl, benzamacril, benzamacril-isobutyl, bialaphos, binapacryl, biphenyl, bitertanol, blasticidin-S, bromuconazole, bupirimate, buthiobate, calcium polysulfide, capsimycin, captafol, captan, carbendazim, carboxin, carvon, quinomethionate, chlobenthiazone, chlorfenazole, chloroneb, chloropicrin, chlorothalonil, chlozolinate, clozylacon, cufraneb, cymoxanil, cyproconazole, cyprodinil, cyprofuram, debacarb, dichlorophen, diclobutrazole, diclofluanid, diclomezine, dicloran, diethofencarb, difenoconazole, dimethirimol, dimethomorph, diniconazole, diniconazole-M, dinocap, diphenylamine, dipyrithione, ditalimfos, dithianon, dodemorph, dodine, drazoxolon, edifenphos, epoxiconazole, etaconazole, ethirimol, etridiazole, famoxadon, fenapanil, fenarimol, fenbuconazole, fenfuram, fenitropan, fenpiclonil, fenpropidin, fenpropimorph, fentin acetate, fentin hydroxide, ferbam, ferimzone, fluazinam, flumetover, fluoromide, fluquinconazole, flurprimidol, flusilazole, flusulfamide, flutolanil, flutriafol, folpet, fosetyl-aluminium, fosetyl-sodium, fthalide, fuberidazole, furalaxyl, furametpyr, furcarbonil, furconazole, furconazole-cis, furmecyclox, guazatine, hexachlorobenzene, hexaconazole, hymexazole, imazalil, imibenconazole, iminoctadine, iminoctadine albesilate, iminoctadine triacetate, iodocarb, ipconazole, iprobenfos (IBP), iprodione, irumamycin, isoprothiolane, isovaledione, kasugamycin, kresoxim-methyl, copper preparations, such as: copper hydroxide, copper naphthenate, copper oxychloride, copper sulfate, copper oxide, oxine-copper and Bordeaux mixture, mancopper, mancozeb, maneb, meferimzone, mepanipyrim, mepronil, metalaxyl, metconazole, methasulfocarb, methfuroxam, metiram, metomeclam, metsulfovax, mildiomycin, myclobutanil, myclozolin, nickel dimethyldithiocarbamate, nitrothal-isopropyl, nuarimol, ofurace, oxadixyl, oxamocarb, oxolinic acid, oxycarboxim, oxyfenthiin, paclobutrazole, pefurazoate, penconazole, pencycuron, phosdiphen, pimaricin, piperalin, polyoxin, polyoxorim, probenazole, prochloraz, procymidone, propamocarb, propanosine-sodium, propiconazole, propineb, pyrazophos, pyrifenox, pyrimethanil, pyroquilon, pyroxyfur, quinconazole, quintozene (PCNB), sulfur and sulfur preparations, tebuconazole, tecloftalam, tecnazene, tetcyclacis, tetraconazole, thiabendazole, thicyofen, thifluzamide, thiophanate-methyl, thiram, tioxymid, toiclofos-methyl, tolylfluanid, triadimefon, triadimenol, triazbutil, triazoxide, trichlamide, tricyclazole, tridemorph, triflumizole, triforine, triticonazole, uniconazole, validamycin A, vinclozolin, viniconazole, zarilamide, zineb, ziram and also Dagger G, OK-8705, OK-8801, α-(1,1-dimethylethyl)-β-(2-phenoxyethyl)-1H-1,2,4-triazole-1-ethanol, α-(2,4-dichlorophenyl)-β-fluoro-β-propyl-1H-1,2,4-triazole-1-ethanol, α-(2,4-dichlorophenyl)-β-methoxy-β-methyl-1H-1,2,4-triazole-1-ethanol, α-(5-methyl-1,3-dioxan-5-yl)-β-[[4-(trifluoromethyl)-phenyl]-methylene]-1H-1,2,4-triazole-1-ethanol, (5RS,6RS)-6-hydroxy-2,2,7,7-tetramethyl-5-(1H-1,2,4-triazol-1-yl)-3-octanone, (E)-α-(methoxyimino)-N-methyl-2-phenoxyphenylacetamide, 1-isopropyl {2-methyl-1-[[[1-(4-methylphenyl)-ethyl]-amino]-carbonyl]-propyl}-carbamate, 1-(2,4-dichlorophenyl)-2-(1H-1,2,4-triazol-1-yl)-ethanone-O-(phenylmethyl)-oxime, 1-(2-methyl-1-naphthalenyl)-1H-pyrrol-2,5-dione, 1-(3,5-dichlorophenyl)-3-(2-propenyl)-2,5-pyrrolidindione, 1-[(diiodomethyl)-sulfonyl]-4-methyl-benzene, 1-[[2-(2,4-dichlorophenyl)-1,3-dioxolan-2-yl]-methyl]-1H-imidazole, 1-[[2-(4-chlorophenyl)-3-phenyloxiranyl]-methyl]-1H-1,2,4-triazole, 1-[1-[2-[(2,4-dichlorophenyl)-methoxy]-phenyl]-ethenyl]-1H-imidazole, 1-methyl-5-nonyl-2-(phenylmethyl)-3-pyrrolidinole, 2′,6′-dibromo-2-methyl-4′-trifluoromethoxy-4′-trifluoro-methyl-1,3-thiazole-5-carboxanilide, 2,2-dichloro-N-[1-(4-chlorophenyl)-ethyl]-1-ethyl-3-methyl-cyclopropanecarboxamide, 2,6-dichloro-5-(methylthio)-4-pyrimidinyl-thiocyanate, 2,6-dichloro-N-(4-trifluoromethylbenzyl)-benzamide, 2,6-dichloro-N-[[4-(trifluoromethyl)-phenyl]-methyl]-benzamide, 2-(2,3,3-triiodo-2-propenyl)-2H-tetrazole, 2-[(1-methylethyl)sulfonyl]-5-(trichloromethyl)-1,3,4-thiadiazole, 2-[[6-deoxy-4-O-(4-O-methyl-β-D-glycopyranosyl)-α-D-glucopyranosyl]-amino]-4-methoxy-1H-pyrrolo[2,3-d]pyrimidine-5-carbonitrile, 2-aminobutane, 2-bromo-2-(bromomethyl)-pentanedinitrile, 2-chloro-N-(2,3-dihydro-1, 1,3-trimethyl-1H-inden-4-yl)-3-pyridinecarboxamide, 2-chloro-N-(2,6-dimethylphenyl)-N-(isothiocyanatomethyl)-acetamide, 2-phenylphenol (OPP), 3,4-dichloro-1-[4-(difluoromethoxy)-phenyl]-1H-pyrrol-2,5-dione, 3,5-dichloro-N-[cyano-[(1-methyl-2-propynyl)-oxy]-methyl]-benzamide, 3-(1,1-dimethylpropyl)-1-oxo-1H-indene-2-carbonitrile, 3-[2-(4-chlorophenyl)-5-ethoxy-3-isoxazolidinyl]-pyridine, 4-chloro-2-cyano-N,N-dimethyl-5-(4-methylphenyl)-1H-imidazole-1-sulfonamide, 4-methyl-tetrazolo[1,5-a]quinazolin-5(4H)-one, 8-(1,1-dimethylethyl)-N-ethyl-N-propyl-1,4-dioxaspiro[4.5]decane-2-methaneamine, 8-hydroxyquinoline sulfate, 9H-xanthene-2-[(phenylamino)-carbonyl]-9-carboxylic hydrazide, bis-(1-methylethyl)-3-methyl-4-[(3-methylbenzoyl)-oxy]-2,5-thiophenedicarboxylate, cis-1-(4-chlorophenyl)-2-(1H-1,2,4-triazol-1-yl)-cycloheptanol, cis-4-[3-[4-(1,1-dimethylpropyl)-phenyl-2-methylpropyl]-2,6-dimethylmorpholinehydrochloride, ethyl [(4-chlorophenyl)-azo]-cyanoacetate, potassium hydrogen carbonate, methanetetrathiol sodium salt, methyl 1-(2,3-dihydro-2,2-dimethyl-1H-inden-1-yl)-1H-imidazole-5-carboxylate, methyl N-(2,6-dimethylphenyl)-N-(5-isoxazolylcarbonyl)-DL-alaninate, methyl N-(chloroacetyl)-N-(2,6-dimethylphenyl)-DL-alaninate, N-(2,3-dichloro-4-hydroxyphenyl)-1-methyl-cyclohexanecarboxamide, N-(2,6-dimethylphenyl)-2-methoxy-N-(tetrahydro-2-oxo-3-furanyl)-acetamide, N-(2,6-dimethylphenyl)-2-methoxy-N-(tetrahydro-2-oxo-3-thienyl)-acetamide, N-(2-chloro-4-nitrophenyl)-4-methyl-3-nitro-benzenesulfonamide, N-(4-cyclohexylphenyl)-1,4,5,6-tetrahydro-2-pyrimidinamine, N-(4-hexylphenyl)-1,4,5,6-tetrahydro-2-pyrimidinamine, N-(5-chloro-2-methylphenyl)-2-methoxy-N-(2-oxo-3-oxazolidinyl)-acetamide, N-(6-methoxy)-3-pyridinyl)-cyclopropanecarboxamide, N-[2,2,2-trichloro-1-[(chloroacetyl)-amino]-ethyl]-benzamide, N-[3-chloro-4,5-bis(2-propynyloxy)-phenyl]-N′-methoxy-methane-imidamide, N-formyl-N-hydroxy-DL-alanine-sodium salt, O,O-diethyl [2-(dipropylamino)-2-oxoethyl]-ethylphosphoramidothioate, O-methyl S-phenyl phenylpropylphosphoramidothioate, S-methyl 1,2,3-benzothiadiazole-7-carbothioate, spiro[2H]-1-benzopyrane-2,1′(3′H)-isobenzofuran]-3′-one,

Bactericides:

bronopol, dichlorophen, nitrapyrin, nickel dimethyldithiocarbamate, kasugamycin, octhilinone, furancarboxylic acid, oxytetracyclin, probenazole, streptomycin, tecloftalam, copper sulfate and other copper preparations.

Insecticides/Acaricides/Nematicides:

abamectin, acephate, acrinathrin, alanycarb, aldicarb, alphamethrin, amitraz, avermectin, AZ 60541, azadirachtin, azinphos A, azinphos M, azocyclotin, Bacillus thuringiensis, 4-bromo-2-(4-chlorophenyl)-1-(ethoxymethyl)-5-(tri-fluoromethyl)-1H-pyrrole-3-carbonitrile, bendiocarb, benfuracarb, bensultap, betacyfluthrin, bifenthrin, BPMC, brofenprox, bromophos A, bufencarb, buprofezin, butocarboxim, butylpyridaben, cadusafos, carbaryl, carbofuran, carbophenothion, carbosulfan, cartap, chloethocarb, chlorethoxyfos, chlorfenapyr, chlorfenvinphos, chlorfluazuron, chlormephos, N-[(6-chloro-3-pyridinyl)-methyl]-N′-cyano-N-methyl-ethanimidamide, chlorpyrifos, chlorpyrifos M, cis-resmethrin, clocythrin, clofentezine, cyanophos, cycloprothrin, cyfluthrin, cyhalothrin, cyhexatin, cypermethrin, cyromazine, deltamethrin, demeton M, demeton S, demeton S-methyl, diafenthiuron, diazinon, dichlofenthion, dichlorvos, dicliphos, dicrotophos, diethion, diflubenzuron, dimethoate, dimethylvinphos, dioxathion, disulfoton, edifenphos, emamectin, esfenvalerate, ethiofencarb, ethion, ethofenprox, ethoprophos, etrimphos, fenamiphos, fenazaquin, fenbutatin oxide, fenitrothion, fenobucarb, fenothiocarb, fenoxycarb, fenpropathrin, fenpyrad, fenpyroximate, fenthion, fenvalerate, fipronil, fluazinam, fluazuron, flucycloxuron, flucythrinate, flufenoxuron, flufenprox, fluvalinate, fonophos, formothion, fosthiazate, fubfenprox, furathiocarb, HCH, heptenophos, hexaflumuron, hexythiazox, imidacloprid, iprobenfos, isazophos, isofenphos, isoprocarb, isoxathion, ivermectin, lambda-cyhalothrin, lufenuron, malathion, mecarbam, mevinphos, mesulfenphos, metaldehyde, methacrifos, methamidophos, methidathion, methiocarb, methomyl, metolcarb, milbemectin, monocrotophos, moxidectin, naled, NC 184, nitenpyram, omethoate, oxamyl, oxydemethon M, oxydeprofos, parathion A, parathion M, permethrin, phenthoate, phorate, phosalone, phosmet, phosphamidon, phoxim, pirimicarb, pirimiphos M, pirimiphos A, profenofos, promecarb, propaphos, propoxur, prothiofos, prothoate, pymetrozin, pyrachlophos, pyridaphenthion, pyresmethrin, pyrethrum, pyridaben, pyrimidifen, pyriproxifen, quinalphos, salithion, sebufos, silafluofen, sulfotep, sulprofos, tebufenozide, tebufenpyrad, tebupirimiphos, teflubenzuron, tefluthrin, temephos, terbam, terbufos, tetrachlorvinphos, thiafenox, thiodicarb, thiofanox, thiomethon, thionazin, thuringiensin, tralomethrin, triarathen, triazophos, triazuron, trichlorfon, triflumuron, trimethacarb, vamidothion, XMC, xylylcarb, zetamethrin.

It is also possible to admix other known active compounds, such as herbicides, fertilizers and growth-regulating substances.

The following co-components for mixtures, for example, are particularly advantageous for applications in the protection of materials.

Triazoles, Such as:

azaconazole, azocyclotin, bitertanol, bromuconazole, cyproconazole, diclobutrazole, difenoconazole, diniconazole, epoxyconazole, etaconazole, fenbuconazole, fenchlorazole, fenethanil, fluquinconazole, flusilazole, flutriafol, furconazole, hexaconazole, imibenconazole, ipconazole, isozofos, myclobutanil, metconazole, paclobutrazole, penconazole, propioconazole, (±)-cis-1-(4-chlorophenyl)-2-(1H-1,2,4-triazol-1-yl)-cycloheptanol, 2-(1-tert-butyl)-1-(2-chlorophenyl)-3-(1,2,4-triazol-1-yl)-propan-2-ol, tebuconazole, tetraconazole, triadimefon, triadimenol, triapenthenol, triflumizole, triticonazole, uniconazole and their metal salts and acid adducts;

Imidazoles, Such as:

clotrimazole, bifonazole, climbazole, econazole, fenapamil, imazalil, isoconazole, ketoconazole, lombazole, miconazole, pefurazoate, prochloraz, triflumizole, thiazolcar, 1-imidazolyl-1-(4′-chlorophenoxy)-3,3-dimethylbutan-2-one and their metal salts and acid adducts;

Pyridines and Pyrimidines, Such as:

ancymidol, buthiobate, fenarimol, nuarimol, triamirol;

Succinate Dehydrogenase Inhibitors, Such as:

benodanil, carboxim, carboxim sulfoxide, cyclafluramide, fenfuram, flutanil, furcarbanil, furmecyclox, mebenil, mepronil, methfuroxam, metsulfovax, pyrocarbolid, oxycarboxin, shirlan, seedvax;

Naphthalene Derivatives, Such as:

terbinafine, naftifine, butenafine, 3-chloro-7-(2-aza-2,7,7-trimethyl-oct-3-en-5-ine);

Sulfenamides, Such as:

dichlorfluanid, tolylfluanid, folpet, fluorfolpet; captan, captofol;

Benzimidazoles, Such as:

carbendazim, benomyl, fuberidazole, thiabendazole or their salts;

Morpholine Derivatives, Such as:

aldimorph, dimethomorph, dodemorph, falimorph, fenpropidin, enpropimorph, tridemorph, trimorphamide and their arylsulfonates, such as p-toluenesulfonic acid and p-dodecylphenyl-sulfonic acid;

Benzothiazoles, Such as:

2-mercaptobenzothiazole;

Benzothiophene Dioxides, Such as:

N-cyclohexyl-benzo[b]thiophenecarboxamide S,S-dioxide;

Benzamides, Such as:

2,6-dichloro-N-(4-trifluoromethylbenzyl)-benzamide, tecloftalam;

Boron Compounds, Such as:

boric acid, boric acid esters, borax;

Formaldehyde and Formaldehyde-Releasing Compounds, Such as:

benzyl alcohol mono-(poly)-hemiformal, n-butanol hemiformal, dazomet, ethyleneglycol hemiformal, hexa-hydro-S-triazines, hexamethylene-tetramine, N-hydroxymethyl-N′-methylthiourea, N-methylolchloro-acetamide, oxazolidine, paraformaldehyde, taurolin, tetrahydro-1,3-oxazine, N-(2-hydroxypropyl)-amine-methanol;

Isothiazolinones, Such as:

N-methylisothiazolin-3-one, 5-chloro-N-methylisothiazolin-3-one, 4,5-dichloro-N-octylisothiazolin-3-one, 5-chloro-N-octylisothiazolinone, N-octylisothiazolin-3-one, 4,5-trimethylene-isothiazolinones, 4,5-benzisothiazolinones;

Aldehydes, Such as:

cinnamaldehyde, formaldehyde, glutaraldehyde, β-bromocinnamaldehyde;

Thiocyanates, Such as:

Thiocyanatomethylthiobenzothiazole, methylenebisthiocyanate;

Quaternary Ammonium Compounds and Guanidines, Such as:

benzalkonium chloride, benzyldimethyltetradecylammonium chloride, benzyldimethyldodecylammonium chloride, dichlorobenzyl-dimethyl-alkyl-ammonium chloride, didecyidimethylammonium chloride, dioctyl-dimethyl-ammonium chloride, N-hexadecyl-trimethyl-ammonium chloride, 1-hexadecyl-pyridinium chloride, iminoctadine-tris(albesilate);

Iodine Derivatives, Such as:

diiodomethyl p-tolylsulfone, 3-iodo-2-propynyl alcohol, 4-chlorophenyl-3-iodopropargyl formal, 3-bromo-2,3-diiodo-2-propenyl ethylcarbamate, 2,3,3-triiodoallyl alcohol, 3-bromo-2,3-diiodo-2-propenyl alcohol, 3-iodo-2-propynyl n-butylcarbamate, 3-iodo-2-propynyl n-hexylcarbamate, 3-iodo-2-propynyl cyclohexylcarbamate, 3-iodo-2-propynyl phenylcarbamate;

Phenols, Such as:

tribromophenol, tetrachlorophenol, 3-methyl-4-chlorophenol, 3,5-dimethyl-4-chlorophenol, phenoxyethanol, dichlorophen, 2-benzyl-4-chlorophenol, 5-chloro-2-(2,4-dichlorophenoxy)-phenol, hexachlorophene, p-hydroxybenzoic ester, o-phenylphenol, m-phenylphenol, p-phenylphenol and their alkali metal and alkaline earth metal salts;

Microbicides Having an Activated Halogen Group, Such as:

bronopol, bronidox, 2-bromo-2-nitro-1,3-propanediol, 2-bromo-4′-hydroxy-acetophenone, 1-bromo-3-chloro-4,4,5,5-tetramethyl-2-imidazolidinone, β-bromo-β-nitrostyrene, chloroacetamide, chloramine T, 1,3-dibromo-4,4,5,5-tetramethyl-2-imidazolidinone, dichloramine T, 3,4-dichloro-(3H)-1,2-dithiol-3-one, 2,2-dibromo-3-nitrile-propionamide, 1,2-dibromo-2,4-dicyanobutane, halane, halazone, mucochloric acid, phenyl-(2-chlorocyano-vinyl)sulfone, phenyl-(1,2-dichloro-2-cyanovinyl)sulfone, trichloroisocyanuric acid;

Pyridines, Such as:

1-hydroxy-2-pyridinethione (and their Na, Fe, Mn, Zn salts), tetrachloro-4-methylsulfonylpyridine, pyrimethanol, mepanipyrim, dipyrithion, 1-hydroxy-4-methyl-6-(2,4,4-trimethylpentyl)-2(1H)-pyridine;

Methoxyacrylates or the Like, Such as:

azoxystrobin, methyl (E)-methoximino [α-(o-tolyloxy)-o-tolyl]acetate, (E)-2-methoxyimino-N-methyl-2-(2-phenoxyphenyl)acetamide, (E)-2-{2-[6-(2-cyanophenoxy)pyrimid in-4-yloxy]phenyl}-3-methoxyacrylate, O-methyl-2-[([3-methoximino-2-butyl)imino]oxy)o-tolyl]-2-methoximino-acetimidate, 2-[[[[1-(2,5-dimethylphenyl)ethylidene]amino]oxy]methyl]-α-(methoxyimino)-N-methyl-benzeneacetamide, α-(methoxyimino)-N-methyl-2-[[[[1-[3-(trifluoromethyl)phenyl]ethylidene]amino]oxy]methyl]-benzeneacetamide, trifloxystrobin, methyl α-(methoxymethylene)-2-[[[[1-[3-(trifluoromethyl)phenyl]ethylidene]amino]oxy]methyl]-benzeneacetate, 2-[[[5-chloro-3-(trifluoromethyl)-2-pyridinyl]oxy]methyl]-α-(methoxyimino)-N-methyl-benzeneacetamide, methyl 2-[[[cyclopropyl[(4-ethoxyphenyl) imino]methyl]thio]methyl]-α-(methoxyimino)-benzeneacetate, α-(methoxy-imino)-N-methyl-2-(4-methyl-5-phenyl-2,7-dioxa-3,6-diazaocta-3,5-dien-1-yl)-benzeneacetamide, methyl α-(methoxymethylene)-2-(4-methyl-5-phenyl-2,7-dioxa-3,6-diazaocta-3,5-dien-1-yl)-benzeneacetate, α(methoxyimino)-N-methyl-2-[[[1-[3-(trifluoromethyl)phenyl]ethoxy]imino]-methyl]-benzeneacetamide, 2-[[(3,5-dichloro-2-pyridinyl)oxy]methyl]-α-(methoxyimino)-N-methyl-benzeneacetamide, methyl 2-[4,5-dimethyl-9-(4-morpholinyl)-2,7-dioxa-3,6-diazanona-3,5-dien-1-yl]-α-(methoxy-methylene)-benzeneacetate, kresoxim-methyl;

Metal Soaps, Such as:

tin naphthenate, copper naphthenate, zinc naphthenate, tin octoate, copper octoate, zinc octoate, tin 2-ethylhexanoate, copper 2-ethylhexanoate, zinc 2-ethylhexanoate, tin oleate, copper oleate, zinc oleate, tin phosphate, copper phosphate, zinc phosphate, tin benzoate, copper benzoate, zinc benzoate;

Metal Salts, Such as:

copper hydroxycarbonate, sodium dichromate, potassium dichromate, potassium chromate, copper sulfate, copper chloride, copper borate, zinc fluorosilicate, copper fluorosilicate;

Oxides, Such as:

Tributyltin oxide, Cu₂O, CuO, ZnO;

Dithiocarbamates, Such as:

cufraneb, ferban, potassium N-hydroxymethyl-N′-methyl-dithiocarbamate, Na or K dimethyldithiocarbamate, macozeb, maneb, metam, metiram, thiram, zineb, ziram;

Nitriles, Such as:

2,4,5,6-tetrachloroisophthalodinitrile, disodium cyano-dithioimidocarbamate;

Quinolines, Such as:

8-hydroxyquinoline and its copper salts;

Other Fungicides and Bactericides, Such as:

5-hydroxy-2(5H)-furanone; 4,5-benzodithiazolinone, 4,5-trimethylenedithiazolinone, N-(2-p-chlorobenzoylethyl)-hexaminium chloride, 2-oxo-2-(4-hydroxy-phenyl)acet-hydroximic chloride, tris-N-(cyclohexyldiazeniumdioxy)-aluminium, N-(cyclo-hexyldiazeniumdioxy)-tributyltin or K salts, bis-N-(cyclohexyldiazenium-dioxy)-copper, iprovalicarb, fenhexamide, spiroxamine, carpropamide, diflumetorin, quinoxyfen, famoxadone, polyoxorim, acibenzolar-S-methyl, furametpyr, thifluzamide, methalaxyl-M, Ag, Zn or Cu-containing zeolites alone or enclosed in polymeric materials.

Very particularly preferred mixtures are those with azaconazole, bromuconazole, cyproconazole, dichlobutrazole, diniconazole, hexaconazole, metaconazole, penconazole, propiconazole, tebuconazole, dichlofluanid, tolylfluanid, fluorfolpet, methfuroxam, carboxin, N-cyclohexyl-benzo[b]-thiophenecarboxamide S,S-dioxide, fenpiclonil, 4-(2,2-difluoro-1,3-benzodioxol-4-yl)-1H-pyrrol-3-carbonitrile, butenafine, imazalil, N-methyl-isothiazolin-3-one, 5-chloro-N-methylisothiazolin-3-one, N-octylisothiazolin-3-one, dichloro-N-octylisothiazolinone, mercaptobenthiazole, thiocyanatomethylthiobenzothiazole, benzisothiazolinone, N-(2-hydroxypropyl)-amino-methanol, benzyl alcohol (hemi)-formal, N-methylolchloroacetamide, N-(2-hydroxypropyl)-amine-methanol, glutaraldehyde, omadine, dimethyl dicarbonate, 2-bromo-2-nitro-1,3-propanediol and/or 3-iodo-2-propynyl n-butylcarbamate.

Furthermore, in addition to the abovementioned fungicides and bactericides, highly active mixtures are also prepared with other active compounds:

Insecticides/Acaricides/Nematicides:

abamectin, acephat, acetamiprid, acrinathrin, alanycarb, aldicarb, aldoxycarb, aldrin, allethrin, alpha-cypermethrin, amitraz, avermectin, AZ 60541, azadirachtin, azinphos A, azinphos M, azocyclotin, Bacillus thuringiensis, barthrin, 4-bromo-2(4-chlorophenyl)-1-(ethoxymethyl)-5-(trifluoromethyl)-1H-pyrrole-3-carbonitrile, bendiocarb, benfuracarb, bensultap, betacyfluthrin, bifenthrin, bioresmethrin, bioallethrin, bromophos A, bromophos M, bufencarb, buprofezin, butathiophos, butocarboxim, butoxycarboxim, cadusafos, carbaryl, carbofuran, carbophenothion, carbosulfan, cartap, quinomethionate, cloethocarb, chlordane, chlorethoxyfos, chlorfenapyr, chlorfenvinphos, chlorfluazuron, chlormephos, N-[(6-chloro-3-pyridinyl)-methyl]-N′-cyano-N-methyl-ethane-imidamide, chlorpicrin, chlorpyrifos A, chlorpyrifos M, cis-resmethrin, clocythrin, cypophenothrin, clofentezin, coumaphos, cyanophos, cycloprothrin, cyfluthrin, cyhalothrin, cyhexatin, cypermethrin, cyromazin, decamethrin, deltamethrin, demeton M, demeton S, demeton-S-methyl, diafenthiuron, dialiphos, diazinon, 1,2-dibenzoyl-1-(1,1-dimethyl)-hydrazine, DNOC, dichlofenthion, dichlorvos, dicliphos, dicrotophos, difethialone, diflubenzuron, dimethoate, dimethyl-(phenyl)-silyl-methyl 3-phenoxybenzyl ether, dimethyl-(4-ethoxyphenyl)-silylmethyl-3-phenoxybenzyl ether, dimethylvinphos, dioxathion, disulfoton, eflusilanate, emamectin, empenthrin, endosulfan, EPN, esfenvalerate, ethiofencarb, ethion, ethofenprox, etrimphos, etoxazole, etobenzanid, fenamiphos, fenazaquin, fenbutatin oxide, fenfluthrin, fenitrothion, fenobucarb, fenothiocarb, fenoxycarb, fenpropathrin, fenpyrad, fenpyroximat, fensulfothion, fenthion, fenvalerate, fipronil, fluazuron, flucycloxuron, flucythrinate, flufenoxuron, flupyrazotos, flufenzine, flumethrin, flufenprox, fluvalinate, fonophos, formethanate, formothion, fosmethilan fosthiazate, fubfenprox, furathiocarb, halofenocid, HCH, heptenophos, hexaflumuron, hexythiazox, hydramethylnon, hydroprene, imidacloprid, imiprothrin, indoxycarb, iodfenfos, iprinomectin, iprobenfos, isazophos, isoamidophos, isofenphos, isoprocarb, isoprothiolane, isoxathion, ivermectin, lama-cyhalothrin, lufenuron, kadedrin lambda-cyhalothrin, lufenuron, malathion, mecarbam, mervinphos, mesulfenphos, metaldehyde, methacrifos, methamidophos, methidathion, methiocarb, methomyl, metalcarb, milbemectin, monocrotophos, moxiectin, naled, NC₁₈₄, NI 125, nicotine, nitenpyram, omethoate, oxamyl, oxydemethon M, oxydeprofos, parathion A, parathion M, penfluron, permethrin, 2-(4-phenoxyphenoxy)-ethyl ethylcarbamate, phenthoate, phorate, phosalon, phosmet, phosphamidon, phoxim, pirimicarb, pirimiphos M, pirimiphos A, prallethrin, profenophos, promecarb, propaphos, propoxur, prothiophos, prothoate, pymetrozin, pyrachlophos, pyridaphenthion, pyresmethrin, pyrethrum, pyridaben, pyrimidifen, pyriproxifen, pyrithiobac-sodium, quinalphos, resmethrin, RH-7988, rotenone, salithion, sebufos, silafluofen, spinosad, sulfotep, sulprofos, tau-fluvalinate, taroils, tebufenozide, tebufenpyrad, tebupirimphos, teflubenzuron, tefluthrin, temephos, terbam, terbufos, tetrachlorvinphos, tetramethrin, Tetramethacarb, thiacloprid, thiafenox, thiamethoxam, thiapronil, thiodicarb, thiofanox, thiazophos, thiocyclam, thiomethon, thionazin, thuringiensin, tralomethrin, transfluthrin, triarathen, triazophos, triazamate, trichlorfon, triflumuron, trimethacarb, vamidothion, XMC, xylylcarb, zetamethrin;

Molluscicides:

fentin acetate, metaldehyde, methiocarb, niclosamide;

Herbicides and Algicides:

acetochlor, acifluorfen, aclonifen, acrolein, alachlor, alloxydim, ametryn, amidosulfuron, amitrole, ammonium sulfamate, anilofos, asulam, atrazine, azafenidin, aziptrotryne, azimsulfuron, benazolin, benfluralin, benfuresate, bensulfuron, bensulfide, bentazone, benzofencap, benzthiazuron, bifenox, bispyribac, borax, bromacil, bromobutide, bromofenoxim, bromoxynil, butachlor, butamifos, butralin, butylate, bialaphos, benzoyl-prop, bromobutide, butroxydim, carbetamide, carfentrazone-ethyl, carfenstrole, chlomethoxyfen, chloramben, chlorbromuron, chlorflurenol, chloridazon, chlorimuron, chlornitrofen, chloroacetic acid, chloransulam-methyl, cinidon-ethyl, chlorotoluron, chloroxuron, chlorpropham, chlorsulfuron, chlorthal, chlorthiamid, cinmethylin, cinofulsuron, clefoxydim, clethodim, clomazone, chlomeprop, clopyralid, cyanamide, cyanazine, cycloate, cycloxydim, chloroxynil, clodinafop-propargyl, cumyluron, CGA 248757, clometoxyfen, cyhalofop, cyhalofop-butyl, clopyrasuluron, cyclosulfamuron, diclosulam, dichlorprop, dichlorprop-P, diclofop, diethatyl, difenoxuron, difenzoquat, diflufenican, diflufenzopyr, dimefuron, dimepiperate, dimethachlor, dimethipin, dinitramine, dinoseb, dinoseb acetate, dinoterb, diphenamid, dipropetryn, diquat, dithiopyr, diduron, DNOC, DSMA, 2,4-D, daimuron, dalapon, dazomet, 2,4-DB, desmedipham, desmetryn, dicamba, dichlobenil, dimethamid, dithiopyr, dimethametryn, eglinazine, endothal, EPTC, esprocarb, ethalfluralin, ethidimuron, ethofumesate, ethobenzanid, ethoxyfen, ET 751, ethametsulfuron, ethoxysulfuron, fenoxaprop, fenoxaprop-P, fenuron, flamprop, flamprop-M, flazasulfuron, fluazifop, fluazifop-P, fuenachlor, fluchloralin, flufenacet, flumeturon, fluorocglycofen, fluoronitrofen, flupropanate, flurenol, fluridone, flurochloridone, fluroxypyr, fomesafen, fosamine, fosametine, flamprop-isopropyl, flamprop-isopropyl-L, flumiclorac-pentyl, flumipropyn, flumioxzim, flurtamone, flupyrsulfuronmethyl, glyphosate, glufosinate-ammonium haloxyfop, hexazinone, imazamethabenz, isoproturon, isoxaben, isoxapyrifop, imazapyr, imazaquin, imazethapyr, ioxynil, isopropalin, imazosulfuron, imazomox, isoxaflutole, imazapic, KUH 911, KUH 920, lactofen, lenacil, linuron, LS 830556, MCPA, MCPA-thioethyl, MCPB, mecoprop, mecoprop-P, mefenacet, mefluidide, metam, metamitron, metazachlor, methabenzthia-zuron, methazole, methoropytryne, methyidymron, methyl isothiocyanate, metobromuron, metoxuron, metribuzin, metsulfuron, molinate, manolide, monolinuron, MSMA, metolachlor, metosulam, metobenzuron, naproanilide, napropamide, naptalam, neburon, nicosulfuron, norflurazon, sodium chlorate, oxadiazon, oxyfluorfen, oxysulfuron, orbencarb, oryzalin, oxadiargyl, propyzamide, prosulfocarb, pyrazolate, pyrazolsulfuron, pyrazoxyfen, pyribenzoxium, pyributicarb, pyridate, paraquat, pebulate, pendimethalin, pentachlorophenol, pentoxazone, pentanochlor, petroleum oils, phenmedipham, picloram, piperophos, pretilachlor, primisulfuron, prodiamine, prometryn, propachlor, propanil, propaquizafob, propazine, propham, proisochlor, pyriminobac-methyl, pelargonic acid, pyrithiobac, quinmerac, quinocloamine, quizalofop, quizalofop-P, quinchlorac, rimsulfuron, sethoxydim, sifuron, simazine, simetryn, sulfosulfuron, sulfometuron, sulfentrazone, sulcotrione, sulfosate, tar oils, TCA, tebutam, tebuthiuron, terbacil, terbumeton, terbuthylazine, terbutryn, thiazafluoron, thifensulfuron, thiobencarb, thiocarbazil, tralkoxydim, tri-allate, triasulfuron, tribenuron, triclopyr, tridiphane, trietazine, trifluoralin, tycor, thdiazimin, thiazopyr, triflusulfuron, vernolate.

The active compounds can be used as such or in the form of their commercial formulations or the use forms prepared therefrom, such as ready-to-use solutions, suspensions, wettable powders, pastes, soluble powders, dusts and granules. They are used in the customary manner, for example by pouring, spraying, atomizing, spreading, dusting, foaming, brushing on and the like. It is further possible to apply the active compounds by the ultra-low volume method or to inject the active compound formulation, or the active compound itself, into the soil. The seeds of the plants can also be treated.

When the active compounds according to the invention are used as fungicides, it is possible to vary the application rates within a relatively large range, depending on the type of application. In the treatment of parts of plants, the application rates of the active compound are, in general, from about 0.1 to about 10 000 g/ha, preferably from about 10 to about 1000 g/ha. In the treatment of seed, amounts of active compound of from 0.001 to 50 g, from about 0.01 to about 10 g, are generally required per kilogram of seed. In the treatment of the soil, active compound application rates are from about 0.1 to about 10 000 g/ha, preferably from about 1 to about 5000 g/ha.

The compositions used for the protection of industrial materials generally comprise an amount from about 1 to about 95%, preferably from about 10 to about 75%, of the active compounds.

The use concentrations of the active compounds according to the invention depend on the species and the occurrence of the microorganisms to be controlled and on the composition of the material to be protected. The optimal rate of application can be determined by test series. In general, the use concentrations are in the range from about 0.001 to about 5% by weight, preferably from about 0.05 to about 1.0% by weight, based on the material to be protected.

The invention is further described in the following illustrative examples, in which all parts are by weight unless otherwise indicated. The invention, which is set forth in the foregoing disclosure, is not to be limited either in spirit or scope by these examples. Those skilled in the art will readily understand that known variations of the conditions and processes of the following preparative procedures can be used to prepare these compositions. Unless otherwise noted, all temperatures are degrees Celsius and all parts and percentages are parts by weight and percentages by weight, respectively.

### PREPARATION EXAMPLES

### Example 1

5-bromo-5-nitro-2-(2-thienyl)-1,3-dioxane:

15 g (0.075 mol) of 2-bromo-2-nitropropane-1,3-diol (bronopol), 5.6 g (0.05 mol) of thiophenaldehyde and 21.7 g (0.2 mol) of chlorotrimethylsilane were dissolved in 100 ml of dichloromethane and initially boiled under reflux for 254 h. A further 7.8 g of chlorotrimethylsilane was added, and boiling under reflux was then continued for a further 7 h. After cooling, water was added and the organic phase was washed with a saturated Na₂CO₃ solution, dried, concentrated, stirred with n-hexane and filtered off with suction, and the product was dried. This gave 7.3 g of 5-bromo-5-nitro-2-(2-thienyl)-1,3-dioxane having the physical data stated in Table 1.

### Example 2

5-bromo-2-(5-chloro-2-thienyl)-5-nitro-1,3-dioxane:

17.7 g (0.09 mol) of 2-chloro-5-(dimethoxymethyl)thiophene, 0.3 g of p-toluenesulfonic acid and 18.4 g (0.09 mol) of 2-bromo-2-nitropropane-1,3-(bronopol) were dissolved in 100 ml of toluene, and methanol formed was distilled off at about 100° C.

After cooling, precipitated unreacted bronopol was removed by filtration, the filtrate was concentrated and the residue was chromatographed on silica gel (toluene). This gave 17.4 g (=78% of theory) of 5-bromo-2-(5-chloro-2-thienyl)-5-nitro-1,3-dioxane having the physical data Table 1.

The compounds listed in Table 1 were prepared analogously to the procedures described in Examples 1 and 2 and analogously to the processes mentioned in the description.

TABLE 1
Compounds of the general formula (I).

| Example | | | | |
|---|---|---|---|---|
| No. | R¹ | R² | R³ | Physical data |
| 1 | H | H | H | ¹H-NMR (CDCl₃) δ = 4.25 (2H,d), 5.15 |
| | | | | (2H,d), 5.89 (1H,s), 6.98 (1H), 7.15 (1H), |
| | | | | 7.32 (1H). |
| 2 | H | H | Cl | ¹H-NMR (CDCl₃) δ = 2.21 (3H,s), 4.28 |
| | | | | (2H,d), 5.15 (2H,d), 6.85 (1H,s), 6.78 |
| | | | | (1H), 7.22 (1H,). m.p. = 55-57° C. |
| 3 | H | H | Br | ¹H-NMR (CDCl₃) δ = 4.25 (2H,d), 5.15 |
| | | | | (2H,d), 5.78 (1H,s), 6.86 (1H), 6.92 (2H). |
| 4 | H | H | CH₃ | ¹H-NMR (CDCl₃) δ = 2.45 (3H,s), 4.24 |
| | | | | (2H,d), 5.14 (2H,d), 5.80 (1H,s), 6.62 |
| | | | | (1H), 6.90 (1H). |
| 5 | CH₃ | H | H | ¹H-NMR (CDCl₃) δ = 2.21 (3H,s), 4.28 |
| | | | | (2H,d), 5.15 (2H,d), 6.85 (1H,s), 6.78 |
| | | | | (1H), 7.22 (1H,). |
| 6 | H | Br | H | m.p. = 72-75° C. |
| 7 | H | H | NO₂ | m.p. = 96-102° C. |
| 8 | H | Br | NO₂ | ¹H-NMR (CDCl₃) δ = 4.30 (2H,d), 5.20 |
| | | | | (2H,d), 6.80 (1H,s), 7.10 (1H,s). |
| 9 | H | H | C₂H₅ | m.p. = 77° C. |

### USE EXAMPLE A

To demonstrate the activity against bacteria, the minimum inhibitory concentrations (MICs) of compositions according to the invention were determined:

Active compounds according to the invention were added, at rations of from 0.1 mg/ml to 5000 mg/ml, to a defined Landy agar. After the agar had solidified, it was contaminated with pure cultures of the test organisms listed in Table 2. The MIC was determined after 3 days of storage at 28° C. and 60 to 70% relative atmospheric humidity. The MIC was the lowest concentration of active compound at which there was no colonization whatsoever by the microbial species used, as is stated in the table below.

TABLE 2
Minimum inhibitory concentrations (ppm) of compounds of
the formula (I) according to the invention.

| | Pseudomonas | |
|---|---|---|
| Example No. | aeroginosa | Bacillus subtilis |
| 1 | 100 | <40 |
| 2 | 100 | <40 |
| 7 | 200 | <40 |
| 5-bromo-5-nitro-1,3- | 750 | |
| dioxane from | | |
| US 3772443 | | |

### USE EXAMPLE B

To demonstrate the activity against fungi, the minimum inhibitory concentrations (MICs) of compositions according to the invention were determined: Active compounds according to the invention were added, at concentrations of from 0.1 mg/l to 5000 mg/l, to agar prepared using malt extract. After the agar had solidified, it was contaminated with pure cultures of the test organisms listed in Table 3. The MIC was determined after 2 weeks of storage at 28° C. and 60 to 70% relative atmospheric humidity. The MIC was the lowest concentration of active compound at which there was no colonization whatsoever by the fungal species used, as is stated in Table 3 below.

TABLE 3
Minimum inhibitory concentrations (ppm) of compounds of
the formula (I) according to the invention.

| | Penicillium | Chaetomium | Aspergillus |
|---|---|---|---|
| Example No. | brevicaule | globusum | niger |
| 1 | <40 | 100 | 200 |
| 2 | <40 | <40 | 100 |
| 3 | <40 | 100 | 200 |
| 4 | <40 | 100 | 200 |
| 5 | <40 | <40 | 200 |
| 6 | <40 | 100 | 100 |
| 7 | <40 | 100 | 200 |
| 8 | 200 | 400 | >400 |
| 5-bromo-5-nitro-1,3- | | 500 | 1000 |
| dioxane from US | | | |
| 3772443 | | | |

### USE EXAMPLE C

In vitro test for the ED₅₀ determination of microorganisms.

A methanolic solution of the active compound to be tested, admixed with the emulsifier PS16, was pipetted into the wells of microtitre plates. After the solvent had evaporated, 200 μg of potato-dextrose medium were added to each well.

Beforehand, the medium had been admixed with a suitable of spores or mycelium of the fungus to be tested.

The resulting concentrations of the active compound were 0.1, 1, 10 and 100 ppm. The resulting concentration of the emulsifier was 300 ppm.

The plates were then incubated on a shaker at a temperature of 22° C. for 3-5 days until sufficient growth can be detected in the untreated controls.

Evaluation was carried out photometrically at a wavelength of 620 nm. From the measured data of the different concentrations, the effective dose which causes 50% inhibition of fungal growth compared to the untreated control (ED₅₀) was determined.

TABLE 4
(In vitro test for the ED₅₀ determination of microorganisms)

| Example | Alternaria | Phytophtora | Pyricularia | Ustilago |
|---|---|---|---|---|
| No. | mali | cryptogea | oryzae | avenae |
| 2 | 0.35 | 0.25 | 1.85 | 0.52 |
| 3 | 0.11 | <0.10 | 0.67 | <0.10 |
| 4 | 0.32 | 0.46 | 0.60 | <0.10 |
| 5 | 0.32 | <0.10 | | 0.50 |

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

What is claimed is:

1. A 5-bromo-5-nitro-2-thienyl-1,3-dioxane of the formula (I) 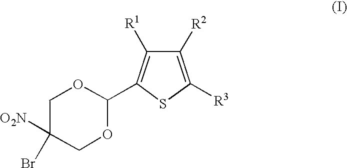
wherein R¹, R² and R³ independently of one another represent hydrogen, halogen, nitro, cyano, or represent in each case an optionally substituted alkyl, alkenyl, alkynyl, acyl, acyloxy, alkoxy, alkoxycarbonyl, alkylthio, amino, mono- or dialkylamino group.

2. The compound according to claim 1, wherein in formula (I), R¹, R² and R³ independently of one another represent (1) hydrogen, (2) halogen, (3) cyano, (4) nitro, or (5) a straight-chain or branched C₁-C₈-alkyl, C₂-C₈-alkenyl or C₂-C₈-alkynyl, which are in each case optionally mono- or polysubstituted by identical or different substitutents from the group consisting of halogen, nitro, and cyano groups, C₁-C₆-alkoxy which is optionally mono- to nonasubstituted by identical or different halogens and C₁-C₆-alkylthio which is optionally mono- to nonasubstituted by identical or different halogens, or (6) a C₁-C₆-acyl, (7) a C₁-C₆-acyloxy, (8) a C₁-C₆-alkoxycarbonyl which is optionally mono- or disubstituted by identical or different halogens, (9) a C₁-C₅-alkoxy, which is optionally mono- to hexasubstituted by identical or different halogens, (10) a C₁-C₅-alkylthio, which is optionally mono- to hexasubstituted by identical or different halogens, or (11) an amino group, (12) a monoalkylamino group having straight-chain or branched C₁-C₆-alkyl radicals or (13) a dialkylamino group having identical or different straight-chain or branched C₁-C₆-alkyl radicals.

3. A process for preparing a 5-bromo-5-nitro-2-thienyl-1,3-dioxane of the formula (I) 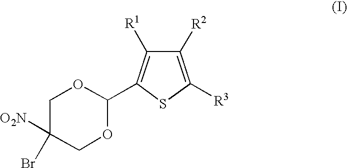
wherein R¹, R² and R³ independently of one another represent hydrogen, halogen, nitro, cyano, or represent in each case an optionally substituted alkyl, alkenyl, alkynyl, acyl, acyloxy, alkoxy, alkoxycarbonyl, alkylthio, amino, mono- or dialkylamino group,
the process comprising:
A) reacting, in a transacetalization reaction, 2-bromo-2-nitropropane-1,3-diol (bronopol) with an acetal of the general formula (III) 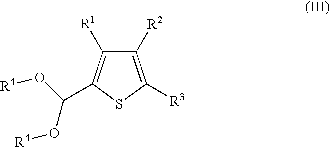
wherein R¹, R² and R³ are independently of one another represent
hydrogen, halogen, nitro, cyano, or represent in each case an optionally substituted alkyl, alkenyl, alkynyl, acyl, acyloxy, alkoxy, alkoxycarbonyl, alkylthio, amino, mono- or dialkylamino group, and
R⁴ represents alkyl, optionally in the presence of a catalyst and optionally, in the presence of a diluent.

4. The process of claim 3, wherein the process is carried out at a temperature ranging from about 10 to about 180° C.

5. The process of claim 3, wherein the process is carried out at a temperature ranging from about 20 and 145° C.

6. A compound of the formula (III): 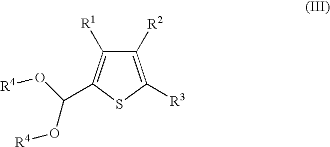
wherein R¹, R² and R³ are independently of one another represent
hydrogen, halogen, nitro, cyano, or represent in each case an optionally substituted alkyl, alkenyl, alkynyl, acyl, acyloxy, alkoxy, alkoxycarbonyl, alkylthio, amino, mono- or dialkylamino group, and
R⁴ represents alkyl.

7. A process for preparing a compound of formula (III) 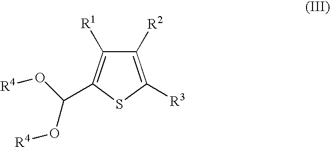
comprising:
(A) reacting (1) an aldehyde of the general formula (II) 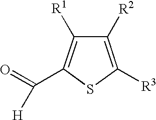
wherein R¹, R² and R³ are independently of one another represent hydrogen, halogen, nitro, cyano, or represent in each case an optionally substituted alkyl, alkenyl, alkynyl, acyl, acyloxy, alkoxy, alkoxycarbonyl, alkylthio, amino, mono- or dialkylamino group, with
(2) an alcohol of the formula (IV), 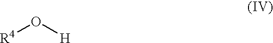
wherein R⁴ represents alkyl,optionally in the presence of a catalyst, optionally in the presence of a dehydrating agent if appropriate, in a diluent.

8. The process of claim 7, wherein the process is carried out at a temperature ranging from about 10 to about 180° C.

9. The process of claim 7, wherein the process is carried out at a temperature ranging from about 20 and 145° C.

6. 10. A method comprising treating an industrial material with a 5-bromo-5-nitro-2-thienyl-1,3-dioxane of the formula (I) 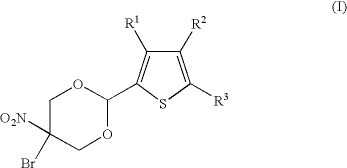
wherein R¹, R² and R³ independently of one another represent hydrogen, halogen, nitro, cyano, or represent in each case an optionally substituted alkyl, alkenyl, alkynyl, acyl, acyloxy, alkoxy, alkoxycarbonyl, alkylthio, amino, mono- or dialkylamino group, and thereby protecting the industrial material against attack and destruction from an microorganism.

11. The method of claim 10, wherein the 5-bromo-5-nitro-2-thienyl-1,3-dioxane of the formula (I) is applied in an amount ranging from about 0.01 to about 5% by weight, based on the engineered material.

12. The method of claim 10, wherein the 5-bromo-5-nitro-2-thienyl-1,3-dioxane of the formula (I) is applied in an amount ranging from about 0.05 to about 1% by weight, based on the engineered material.

13. The method of claim 10, wherein the industrial material is selected from the group consisting of paints, leather, wood, plastics, cooling lubricants, and combinations thereof.

14. A method comprising treating a plant substrate, a seed substrate, or a soil substrate with a 5-bromo-5-nitro-thienyl-1,3-dioxane of the formula (I): 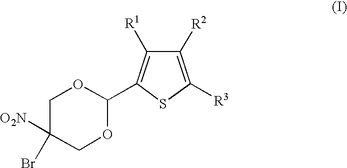
wherein R¹, R² and R³ independently of one another represent hydrogen, halogen, nitro, cyano, or represent in each case an optionally substituted alkyl, alkenyl, alkynyl, acyl, acyloxy, alkoxy, alkoxycarbonyl, alkylthio, amino, mono- or dialkylamino group, and thereby protecting the plant against attack and destruction from an microorganism.

15. The method of claim 14, wherein the 5-bromo-5-nitro-thienyl-1,3-dioxane of the formula (I) is applied in an amount ranging from about 0.1 to about 10,000 ga/hr.

16. The method of claim 14, wherein the 5-bromo-5-nitro-thienyl-1,3-dioxane of the formula (I) is applied in an amount ranging from about 10 to about 1,000 ga/hr.

17. A microbicidal formulation comprising:
(A) a 5-bromo-5-nitro-2-thienyl-1,3-dioxane of the formula (I) 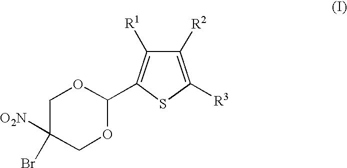
wherein R¹, R² and R³ independently of one another represent hydrogen, halogen, nitro, cyano, or represent in each case an optionally substituted alkyl, alkenyl, alkynyl, acyl, acyloxy, alkoxy, alkoxycarbonyl, alkylthio, amino, mono- or dialkylamino group,
(B) at least one solvent or diluent, and
(C) optionally, a processing auxiliary or an additional antimicrobially active compound.

18. The formulation according to claim 17, wherein the additional antimicrobially active compound is selected from the group consisting of fungicides, bactericides, acaricides, nematicides, algicides, insecticides, and mixtures thereof.

19. The formulation of claim 17, wherein the formulation comprises from about 0.1 to about 95% by weight of active compounds, based on the total weight of the formulation.

20. The formulation of claim 17, wherein the formulation comprises from about 0.5 to about 90%, based on the total weight of the formulation.

21. An industrial material comprising a substrate containing
a 5-bromo-5-nitro-2-thienyl-1,3-dioxane of the formula (I) 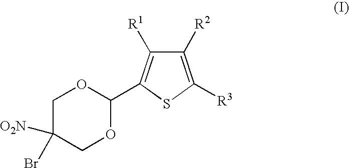
wherein R¹, R² and R³ independently of one another represent hydrogen, halogen, nitro, cyano, or represent in each case an optionally substituted alkyl, alkenyl, alkynyl, acyl, acyloxy, alkoxy, alkoxycarbonyl, alkylthio, amino, mono- or dialkylamino group,
